(19) United States
(12) Patent Application Publication
(10) Pub. No.: US 2025/0046442 A1
(43) Pub. Date: Feb. 6, 2025

(54) STATE OF HEALTH EVALUATION OF ULTRASONIC HANDPIECE

(71) Applicant: Alcon Inc., Fribourg (CH)

(72) Inventors: Guangyao Jia, Irvine, CA (US); Kurt D. Leukanech, Laguna Niguel, CA (US); Satish Yalamanchili, Irvine, CA (US)

(21) Appl. No.: 18/780,763

(22) Filed: Jul. 23, 2024

Related U.S. Application Data
(60) Provisional application No. 63/517,395, filed on Aug. 3, 2023.

(51) Int. Cl. G16H 40/60; A61B 17/00

(52) U.S. Cl. CPC G16H 40/60; A61B 2017/003

(57) ABSTRACT

A controller for an ultrasonic handpiece having a load in the form of at least one piezoelectric drive crystal includes a processor and a computer-readable storage memory on which is recorded a computer-readable instruction set. Execution of the instruction set during real-time operation of the handpiece causes the processor to perform an associated method, during which the processor calculates a load capacitance of the load and a dissipation factor of the load capacitance. The processor also records the load capacitance and dissipation factor in memory over time as a recorded capacitance history. The processor then executes a control action of the handpiece using the recorded capacitance history and possibly one or more additional recorded electrical parameters such as estimated resistance, estimated inductance, and/or measured temperature, or correlations of the same with resonant frequency over time.

The present disclosure relates to methodologies and related automated systems for monitoring the ongoing state of health of an ultrasonic handpiece, e.g., an ultrasonic phacoemulsification tool (“phaco tool”) for use during cataracts surgery.

As appreciated in the art, a variety of factors can cause the proteins of a person's natural lens to break down. The result is a cloudy haze or cataract on the lens, which in turn can impair the person's vision. Removal of the cataract often involves the process of phacoemulsification by which a surgeon directs targeted ultrasonic energy onto the lens. The incident ultrasonic energy, which is emitted from a working tip of the ultrasonic handpiece, fractures the cataractous lens. The resulting lens fragments are then removed from the lens capsule with the assistance of irrigation and suction. Upon removal of the lens fragments, the surgeon will insert an artificial intraocular lens to complete the surgery and thereby restore, and possibly improve, the patient's pre-cataract vision.

### SUMMARY

Disclosed herein are methods and computer-based systems for determining a numeric state of health (SOH) of an ultrasonic handpiece, with the numeric SOH thereafter used in a variety of possible control actions.

As described above, a typical ultrasonic handpiece, also referred to in the art as a phaco tool or probe, provides a reliable mechanism for safely breaking up and extracting a patient's clouded natural lens. Ultrasonic vibration may be induced by activating at least one piezoelectric drive crystal (“piezo-crystals”) encapsulated within an outer housing of the ultrasonic handpiece by applying an electric field to the piezo-crystals. The piezo-crystals act as an electrical energy-to-mechanical energy transducer within the ultrasonic handpiece. However, because of dielectric and mechanical losses, significant heat can also be generated in the piezo-crystals, which in turn can degrade efficiency while potentially shortening the useful lifespan of the ultrasonic handpiece.

Performance and efficiency degradation of the ultrasonic handpiece are also associated with thermal cycling, especially that which is experienced during steam-based sterilization cycles. Moisture ingress from the use of sterilizing steam and due to mechanical stress can also lead to the formation of micro-cracks in the piezo-crystals along with associated efficiency losses. Additionally, prolonged use of the ultrasonic handpiece over many cycles can cause the piezo-crystals to degrade, which in turn can decrease the operating efficiency of the ultrasonic handpiece. The present solutions are therefore intended to optimize the working efficiency and extend the service life of an ultrasonic handpiece using historical information used to determine the above-noted numeric SOH. Additional benefits may include reduced warranty costs and improved overall user satisfaction, e.g., through extended service life of the ultrasonic handpiece.

In accordance with an aspect of the present disclosure, a system for use with an ultrasonic handpiece having the aforementioned piezo-crystals as an internal load includes a processor and memory, e.g., resident within or operatively attached to the ultrasonic handpiece. A computer-readable instruction set is recorded in the memory. Execution of the instruction set by the processor causes the processor to calculate a capacitance value of the ultrasonic handpiece, among other possible values as set forth herein. This action occurs in real-time, i.e., during the ongoing operation of the ultrasonic handpiece.

The capacitance value as contemplated herein includes a clamped capacitance of the aforementioned load, with the clamped capacitance referred to herein as a load capacitance, which itself is an equivalent capacitance of the ultrasonic handpiece arising from mechanical vibration. The capacitance value also include a dissipation factor of the load capacitance as described below. The processor is configured to record the capacitance value in memory over time as a recorded capacitance history. Using this recorded capacitance history and other possible values as part of a larger recorded performance history of the ultrasonic handpiece, the processor could calculate a numeric state of health (SOH) of the load and/or the ultrasonic handpiece, e.g., as a normalized value. The processor may then perform or initiate a control action of the ultrasonic handpiece using the numeric SOH. Various approaches for calculating the numeric SOH are described below.

An aspect of the disclosure includes a controller for use with an ultrasonic handpiece having a load constructed of at least one piezoelectric drive crystal. The controller in this embodiment includes a processor and a computer-readable storage medium, hereinafter a “memory” for simplicity, on which is recorded an instruction set. Execution of the instruction set by the processor causes the processor to calculate each of (i) a load capacitance of the load at a calibrated sampling rate, and (ii) a dissipation factor of the load capacitance during real-time operation of the ultrasonic handpiece. The processor also records the load capacitance and the dissipation factor in the memory as a recorded capacitance history. Additionally, execution of the instruction set causes the controller to calculate a numeric SOH of the load using the recorded capacitance history, and to execute a control action of the ultrasonic handpiece in response to the numeric SOH.

Also disclosed herein is a method for evaluating an ultrasonic handpiece having a load constructed of at least one piezoelectric drive crystal. An embodiment of the method includes calculating, via a processor of a controller, (i) a load capacitance of the load at a calibrated sampling rate, and (ii) a dissipation factor of the load capacitance during real-time operation of the ultrasonic handpiece, wherein the load capacitance and the dissipation factor together form capacitance values. The method also includes recording the capacitance values in the above-noted memory of the controller at the calibrated sampling rate as a recorded capacitance history. Additionally, the method includes calculating a numeric SOH of the load via the processor using the recorded capacitance history, and then executing a control action of the ultrasonic handpiece via the processor in response to the numeric SOH. The control action in this implementation includes outputting an electronic health signal to an external computer device in response to the numeric SOH. The electronic health signal is representative of a relative health level of the load as indicated by the numeric SOH.

Another aspect of the present disclosure includes an ultrasonic handpiece having a housing defining a cavity therein, a working tip connected to the housing, a load contained within the cavity, and the above-summarized controller. The load, which is constructed of at least one piezoelectric drive crystal, vibrates the working tip at a resonant frequency when the load is activated at a drive frequency. The controller is connected to the housing and configured to perform embodiments of the method as summarized above.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is an illustration of a representative ultrasonic handpiece configured as set forth herein.

FIG. 1A illustrates a tip of the ultrasonic handpiece of FIG. 1 in the process of breaking up and removing a cataract from a patient's lens.

FIG. 2 is a schematic illustration of a controller for use with the ultrasonic handpiece of FIG. 1 in accordance with an aspect of the disclosure.

FIG. 3 is an equivalent circuit representing the ultrasonic handpiece of FIG. 1.

FIG. 4 is a flowchart describing a method for determining a numeric state of health (SOH) of the ultrasonic handpiece, and for using the numeric SOH in the performance of one or more control actions as described in detail below.

The foregoing and other features of the present disclosure are more fully apparent from the following description and appended claims, taken in conjunction with the accompanying drawings.

### DETAILED DESCRIPTION

Embodiments of the present disclosure are described herein. It is to be understood, however, that the disclosed embodiments are merely examples and other embodiments can take various and alternative forms. The figures are not necessarily drawn to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

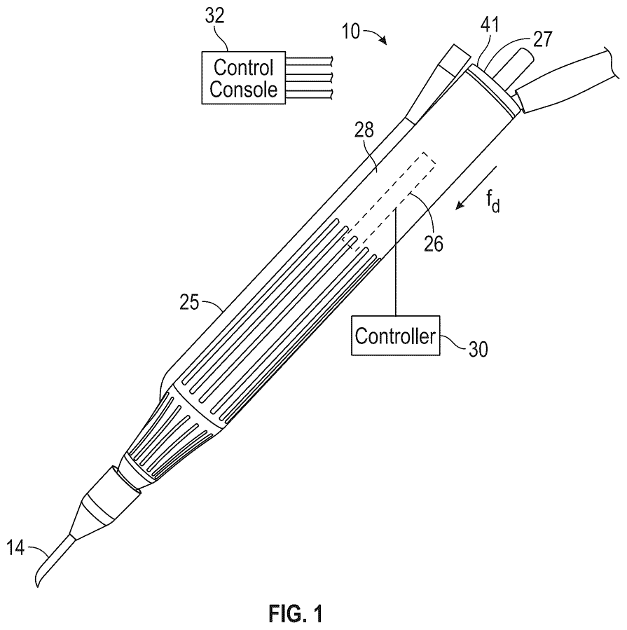

Referring to the drawings, wherein like reference numbers refer to like components, an ultrasonic handpiece 10 is shown in FIG. 1 that is configured to perform a phacoemulsification procedure on a patient's eye 12, an anterior portion 13 of which is depicted in the simplified illustration of FIG. 1A as described below. As appreciated in the art, a working tip 14 of the ultrasonic handpiece 10, also referred to in the art as a phaco probe or tool as noted above, is inserted into the patient's eye 12 and used to break up and remove a formed cataract, e.g., a nuclear sclerotic, cortical, or posterior subcapsular cataract.

Referring briefly to FIG. 1A, during a typical phacoemulsification procedure, a surgeon inserts the working tip 14 of the ultrasonic handpiece 10 through a small incision 15 previously formed in the cornea 16 of the patient's eye 12. The tip 14 is then carefully maneuvered into a capsular bag 18 surrounding a cataractous natural lens 21 just aft of the iris 22. Ultrasonic energy is then emitted from the tip 14 into the lens 21 to thereby fracture the lens 21 and a cataract 20 formed thereon. Incisional compression on the tip 14 and contact of the tip 14 and the lens 21 constitute a load on the ultrasonic handpiece 10 of FIG. 1 within the scope of the disclosure.

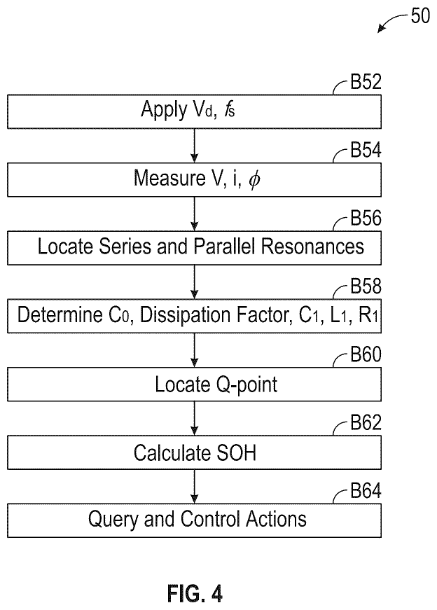

Referring once again to FIG. 1, the ultrasonic handpiece 10 may include an elongated handle or housing 25 connected to or integrally formed with the above-noted tip 14, with the housing 25 configured to be grasped by the surgeon during the procedure The ultrasonic handpiece 10 also includes at least one piezoelectric drive crystal (e.g., one piezoelectric drive crystal or a stack of piezoelectric drive crystals) (“piezo-crystals”) 26 enclosed within a cavity 28 of the housing 25 or packaged within another suitable space within the ultrasonic handpiece 10. As appreciated in the art, the ultrasonic handpieces 10 essentially acts a solid bar that vibrates at a resonant frequency when the piezo-crystals 26 are activated by electrical power from a surgical control console 32. The resulting vibration is amplified at the tip 14 due to its smaller cross-sectional diameter relative to the housing 25. The piezo-crystals 26 thus act as a driven load within the context of the present method 50, a non-limiting representative embodiment of which is described below with reference to FIG. 4.

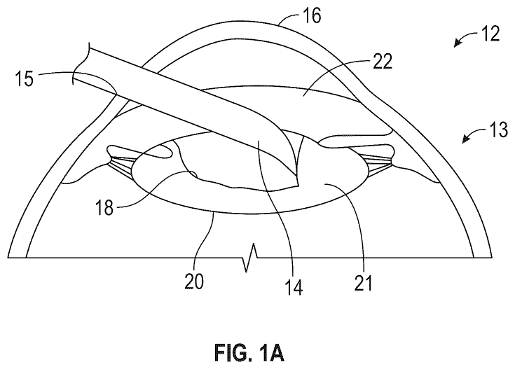

The ultrasonic handpiece 10 illustrated in FIG. 1 also includes or is connected to a controller 30. In a possible implementation, portions of the controller 30, e.g., any or all of the required drive components, an integrated circuit, microelectromechanical system, or a system-on-a-chip, could be connected to an end 27 of the housing 25. For example, components of the controller 30 in a possible construction could be integrated into an external electrical connector 41 configured to connect the ultrasonic handpiece 10 to the surgical control console 32, and thus to electrical power, fluidic power, suction, and/or other surgery-specific functions of the surgical control console 32. Due to limited packaging space aboard the ultrasonic handpiece 10, some or all of components of the controller 30 may be located external to the ultrasonic handpiece 10. For instance, memory 34 of the controller 30 (see FIG. 2), e.g., electrically-erasable programmable read-only memory (EEPROM), could be co-located with the ultrasonic handpiece 10. Other components could likewise be co-located with the ultrasonic handpiece 10 as space permits, for instance a temperature sensor 38T as also shown in FIG. 2, with remaining sensors and processing circuitry possibly being integrated with the surgical control console 32.

Although present technology makes the implementation of this option perhaps less practicable, the controller 30 in some embodiments could be housed fully within the housing 25 shown in FIG. 1, e.g., as a miniaturized system-on-a-chip (SoC). The controller 30 in these and other contemplated embodiments is specially configured to monitor the performance of the ultrasonic handpiece 10 over time during multiple uses, periodically calculate a numeric state of health of the ultrasonic handpiece 10, e.g., at a calibrated sampling rate, and selectively modify a drive frequency (f_{d}) of the ultrasonic handpiece 10 as needed based at least in part on the numeric SOH, its underlying measurements, and possibly other factors.

Referring now to FIG. 2, the controller 30 of FIG. 1 as contemplated herein is programmed in software and equipped in hardware, i.e., “configured”, to execute computer-readable instructions from non-volatile portions of its memory (M) 34 using a processor (P) 36. The memory 34 may include a computer-readable storage medium in the form of tangible non-transitory memory, e.g., optical, magnetic, flash, or other types of read only memory, along with application-sufficient amounts of random-access memory, electrically-erasable programmable read only memory, etc. The processor 36 for its part may be constructed from various combinations of Application Specific Integrated Circuit(s) (ASICs), Field-Programmable Gate Arrays (FPGAs), electronic circuits, central processing units, microprocessors, and the like. Non-transitory components of the memory 34 record or store computer-readable instructions for controlling operation of the ultrasonic handpiece 10 of FIG. 1, including an instruction set for performing a method 50, an exemplary embodiment of which is depicted in FIG. 4 and described in detail below.

In order to perform the various calculations and control actions of the method 50, the controller 30 is also equipped with a sensor suite 38, including, e.g., one or more capacitance sensors 38C, voltage sensors 38V, current sensors 381, temperature sensors 38T, e.g., thermistors, and phase angle sensors 38U. As used herein, “sensor” for any of the sensors of the sensor suite 38 may include one or more physical sensors and associated circuitry needed to output a corresponding value. As noted above, the temperature sensor(s) 38T in one or more embodiments may be connected to or contained within the housing 25 of FIG. 1 or otherwise attached to the ultrasonic handpiece 10. The controller 30 as illustrated in FIG. 2 is thus configured to measure a set of performance data using the sensor suite 38 and report the measured data as sensor signals (CCs) to the processor 36. The processor 36 in turn calculates the numeric SOH of the ultrasonic handpiece 10 of FIG. 1 and the drive frequency (f_{d}) needed for optimum control of the piezo-crystals 26 thereof.

As described below with reference to FIG. 4, the controller 30 in some embodiments may be in communication with an external computer device 44, e.g., via a cloud-based, wireless, or hard-wired network connection 47, with the external computer device 44 being configured as one or more remote cloud-based servers, tablet computers, smart phones, or other application-suitable devices. To that end, the external computer device 44 in some embodiments could selectively transmit a request signal 45 to the controller 30, with the controller 30 receiving the request signal 45 via a radio frequency (RF) antenna 40. In response to receipt of the request signal 45, the controller 30 may transmit the numeric SOH to the external computer device 44 as an output signal 42. The output signal 42 may be optionally embodied in an electronic health signal sent in response to the numeric SOH, determined as set forth below with reference to the method 50 shown in FIG. 4, and representative of a relative health level of the load, i.e., the piezo-crystals 26, as indicated by the numeric SOH. Exemplary embodiments of such an electronic health signal include a text message or an e-mail, or other possible signals or messages.

The external computer device 44 in such an embodiment could be used by a warranty repair depot or service department to ascertain the true remaining useful life of the ultrasonic handpiece 10 of FIG. 1, and possibly to alert a user of the ultrasonic handpiece 10 of the remaining useful life, or to provide advance notice of a predicted timeframe for its replacement. The user in other implementations could be similarly informed as to the numeric SOH, e.g., as a normalized value in the range of 0 to 1, with 0 in this case corresponding to a non-functioning/fully depleted unit and 1 corresponding to a new/properly functioning unit.

Ultrasonic handpieces such as the representative ultrasonic handpiece 10 shown in FIG. 1 are typically factory-calibrated or tuned with an ultrasonic stroke. As the method 50 of FIG. 4 is performed, an optimal drive frequency f_{d} or “Q-point” is determined prior to each use of the ultrasonic handpiece 10 of FIG. 1. To that end, the drive voltage and current are provided, the resonant frequency is determined, and the ultrasonic handpiece 10 is tuned accordingly. Generally, the Q-point is initially selected somewhere between series and parallel resonance points of the particular ultrasonic handpiece being tuned. During ongoing operation, an associated control unit may adjust the drive frequency using the measured voltage and current as feedback values. Initial tuning thus results in initiation of control of the above-noted drive frequency (f_{d}).

Once electrical power has been applied to the ultrasonic handpiece 10, however, the Q-point will begin to vary from its initial setting. This occurs as a function of the internal load, i.e., the piezo-crystals 26, and as a result of self-heating. The present approach therefore also monitors for such changes in the Q-point, including in particular changes related to the clamped load capacitance of the piezo-crystals 26 of FIG. 1 and associated coupling coefficients. The computer-based solutions set forth herein therefore seek to optimize the Q-point in real-time based upon changes in the load capacitance and possibly other values.

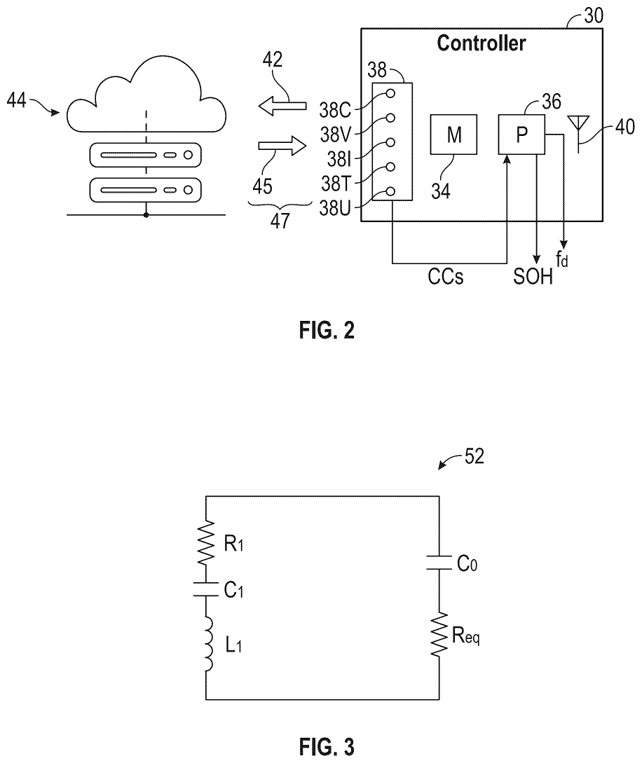

The above concept can be better understood with reference to an equivalent circuit of the ultrasonic handpiece 10, which will now be described with reference to an equivalent circuit 52 as shown in FIG. 3. The equivalent circuit 52 reduces the ultrasonic handpiece 10 of FIG. 1 to its fundamental electrical parameters, including an estimated equivalent internal resistance (R₁), the above-noted load capacitance (C₀) of the piezo-crystals 26, an equivalent internal capacitance (C₁) of the ultrasonic handpiece 10 resulting from mechanical vibration, an estimated equivalent internal inductance (L₁), and an equivalent series resistance (R_{eq}) of the load capacitance (C₀). With respect to the latter, the above-noted dissipation factor (DF) may be represented as DF=2πfC₀R_{eq}, where 2πf is the angular frequency of operation. Each of these electrical values may be determined offline and recorded in memory 34 of the controller 30 shown in FIG. 2 for use in performing the present method 50 of FIG. 4, with the processor 36 updating these values and constructing a history thereof as described below.

The ability of the piezo-crystals 26 (the load) of FIG. 1 to convert applied electrical energy into mechanical energy generally deteriorates over time and with repeated or prolonged use of the ultrasonic handpiece 10. This deterioration is caused by a number of factors such as depolarization, thermal cycling, micro-cracks, and an ingress of water. These and other possible influencing factors impact the load capacitance (C₀), both in terms of magnitude as well as in terms of dissipation factor. The noted parameters tend to remain relatively constant during the life of the ultrasonic handpiece 10. That is, the load capacitance (C₀) is related to the dielectric properties of the piezo-crystals 26, and thus remains approximately constant during operation. However, when the ultrasonic handpiece 10 approaches the end of its useful working life, at least some of these measurables will begin to change, including the load capacitance (C₀) due to aging of the piezo-crystals 26. This can degrade operating performance, possibly leading to reduced efficiency and increased heating. Because such deterioration is gradual, it can be difficult, absent the present teachings, to ascertain precisely when the ultrasonic handpiece 10 has reached the end of its useful life and requires replacement.

Within the scope of the present disclosure, non-linear control of the ultrasound handpiece 10 can be achieved by (i) monitoring the changing load capacitance (C₀) of the piezo-crystals 26, or (ii) by monitoring the load (the piezo-crystals 26) and computing power delivery inefficiency based on heat generation. The controller 30 of FIG. 2 may then adjust the Q-point in real-time using the inefficiency as a feedback value. Doing so would allow for improved efficiency as the load varies during use and due to aging, as noted above.

Near the resonance frequencies, the piezo-crystals 26 can be modeled as the simple equivalent circuit 52 of FIG. 3. Natural frequencies of the piezo-crystals 26, i.e., resonance and anti-resonance frequencies, may be determined by the following equations:

$${f}_{r} = \frac{1}{2 π \sqrt{{L}_{1} {C}_{1}}}$$

$${f}_{a} = \frac{1}{2 π} \sqrt{\frac{{C}_{1} + {C}_{0}}{{L}_{1} {C}_{1} {C}_{0}}}$$

where fᵣ and fₐ represent the resonance and anti-resonance frequencies, respectively. During operation of the ultrasonic handpiece 10 of FIG. 1, the internal resistance (R₁), inductance (L₁), and capacitance (C₁) may change due to self-heating. Variation in the internal inductance (L₁) and capacitance (C₁) will result in shifting of the resonant frequency (fᵣ), while changes to the internal resistance (R₁) in turn will lead to a variation in the electrical impedance at the resonant frequency (fᵣ).

The value of the load capacitance (C₀) monitored herein by the controller 30 is directly related to the particular dielectric properties of the piezo-crystals 26 shown in FIG. 1. The value of the load capacitance (C₀) is subject to change due to operating temperature, which in turn can increase with sustained operation of the ultrasonic handpiece 10. The load capacitance (C₀) is also subject to change due to aging-induced changes in the dielectric properties of the piezo-crystals 26. As noted above, such changes are related to factors such as thermal cycling, depolarization, micro-cracks, and ingress of moisture and other contaminants over time. The load capacitance (C₀) and possibly at least one additional electrical parameter such as the internal capacitance (C₁), inductance (L₁), and/or resistance (R₁) may be used to estimate the current level of degradation of the piezo-crystals 26, and ultimately to determine the numeric SOH of the ultrasonic handpiece 10.

That is, once the load capacitance (C₀) has been determined, an estimate of the values for the equivalent inductance and resistance values L₁ and R₁ can be determined by locating the resonance and anti-resonance frequencies, and by thereafter using the relationships described above. Doing this can provide the initial location of the Q-point. The shift in Q-point can then be determined by measuring the temperature of the piezo-crystals 26. The relationship between crystal temperature and the shift in Q-point may also be predicted by using the historical data of the load capacitance C₀ measurements. In other words, the historical knowledge of the capacitance values contemplated herein gives the age of the piezo-crystals 26, and thus may influence the temperature-to-Q-point relationship. In practical terms, an older version of the ultrasonic handpiece 10 of FIG. 1 is more affected by temperature changes than is a new version of the same ultrasonic handpiece 10.

Additionally, correlations between a shift of natural frequencies and temperature rises due to self-heating of the ultrasonic handpiece 10 of FIG. 1 may be established with the piezo-crystals 26 throughout the service life of the ultrasonic handpiece 10, i.e.:

Δfᵣ=fᵣ(T,N)

Δf_{α}=fᵣ(T,N)

where T is the operating temperature and N represents the number of uses of the ultrasonic handpiece 10. This particular approach thus uses a count of the number of uses of the ultrasonic handpiece 10 to provide the relationship between resonance and temperature. This is just one possible way to determine or estimate the age of the ultrasonic handpiece 10 within the scope of the present disclosure. Such correlations may be used to predict shifts in the natural frequencies by measuring the temperature of the load during operation, e.g., using the temperature sensor 38T of FIG. 2, and thereby adjusting the drive frequency to the piezo-crystals 26 accordingly so as to achieve higher operating efficiencies. The present approach could therefore include establishing correlations between temperature rise and resonant frequency of the ultrasonic handpiece 10 during its operation, e.g., recording a first correlation in the memory 34, and between aging time and resonant frequency of the ultrasonic handpiece 10, e.g., recording a second correlation in the memory 34. Such correlations can then be used to adjust the drive frequency so as to maintain high-efficiency and reduce self-heating of the ultrasonic handpiece 10.

The present approach could also include recording a first correlation between a temperature increase and resonant frequency (fᵣ) of the ultrasonic handpiece 10, recording a second correlation between an aging time and a resonant frequency of the ultrasonic handpiece 10, and selectively adjusting the drive frequency of the load from the control console 32 using the first correlation and the second correlation, via the controller 30. This could occur as at least part of the control action, such that the drive frequency (f_{d}) is moved closer in value to a resonant frequency of the ultrasonic handpiece 10.

Referring now to FIG. 4, an embodiment of the present method 50 commences prior to each operation of the ultrasonic handpiece 10 shown in FIG. 1. The method 50 is illustrated in simplified form as being organized into discrete logic blocks. Each logic block in turn represents a particular step, function, or subprocess that is to be performed via the controller 30 of FIG. 1 when executing the present method 50.

Beginning with block B52, a drive voltage (V_{d}) is applied to the ultrasonic handpiece 10 through a predefined sweep frequency range (fₛ). This occurs upon use of the ultrasonic handpiece 10, e.g., by plugging the ultrasonic handpiece 10 into the surgical control console 32 of FIG. 1 and energizing the ultrasonic handpiece 10. The method 50 then proceeds to block B54.

At block B54, the processor 36 of FIG. 2, continuously or at a calibrated sampling rate, measures the voltage (v), current (i), and phase angle (ϕ) using the sensor suite 38 of FIG. 2. These values are then temporarily recorded in memory 34. The method 50 then proceeds to block B56.

Block B56 includes locating the series and parallel resonances of the ultrasonic handpiece 10 of FIG. 1. The method 50 proceeds to block B58 after completing this computation.

At block B58, the processor 36 of FIG. 2 next calculates and records the load capacitance (C₀) and dissipation factor thereof. In some embodiments, the processor 36 also calculates the mechanical capacitance (C₁) and estimates the inductance (L₁) and the resistance (R₁). The recorded capacitance values in particular, i.e., at least the load capacitance and dissipation factor, are used to form a capacitance history, i.e., a time-stamped sequence of recorded values describing the trajectory of the load capacitance (C₀) and dissipation factor, and possibly the mechanical capacitance (C₁), over an extended number of uses of the ultrasonic handpiece 10, or over an extended period of time of weeks, months, or years. The method 50 proceeds to block B60 after recording these values and forming the capacitance history.

Within the course of the method 50, the values of block B58 are determined each time the ultrasonic handpiece 10 is used, i.e., during a preparatory tuning process. Thus, a history is developed and used to predict the dynamic nature of the above-described Q-point. The controller 30 then updates this history over time. As part of block B58, capacitance could be determined via measurement using the capacitance sensor(s) 38C of FIG. 2, e.g., by measuring the value of C₀ directly by measurement of the overall impedance of the ultrasonic handpiece 10 at a frequency which is sufficiently far from the resonance frequency.

The dissipation factor refers to the power loss occurring across the capacitor when alternating current power is applied to the ultrasonic handpiece 10. The dissipation factor as described above is essentially the amount of loss/resistance of the load capacitance C₀. The dissipation factor is related to the equivalent series resistance (R_{eq}) of the load capacitance C₀. Both values may be determined in real-time and recorded in or on a computer-readable storage medium of the memory 34 depicted in FIG. 2. The resistance (R₁) and inductance (L₁) in turn are estimated as described above.

Still referring to FIG. 4, at block B60, the processor 36 locates the optimal frequency or Q-point as set forth in detail hereinabove. Next, at block B62 the processor 36 calculates the numeric SOH using the values in the recorded capacitance history. The numeric SOH in a possible implementation could entail a ratio of a baseline dissipation factor, e.g., one that is measured or otherwise determined at time of initial manufacturing of the ultrasonic handpiece 10, divided by the latest-computed value of the dissipation factor. The numeric SOH could then be used to perform one or more control action(s) as part of block B62.

For example, the processor 36 could determine the capacitance values (C₀, C₁) and dissipation factor for a newly manufactured and properly tuned ultrasonic handpiece 10, and then associate these values in memory 34 with full health or proper expected function. On a normalized scale, this may equate to a numeric SOH of 1. The processor 36 could then associate other values on the continuum to a numeric SOH ranging from 0 to 1. Such values could be associated in a lookup table, for example, for rapidity and case of access. The method 50 proceeds to block B64 once the numeric SOH has been calculated.

Block B64 may entail receiving, via the processor 36 of FIG. 2, a query from the external computer device 44 of FIG. 2 in the form of the request signal 45. For example, at scheduled or randomly selected times, the external computer device 44 could transmit the request signal 45 to the controller 30 to thereby request transmission of the recorded capacitance history, which resides in the memory 34 due to execution of the method 50. The request signal 45 could also request transmission of the numeric SOH if previously calculated via the processor 36, or the external computer device 44 could calculate the numeric SOH using the reported capacitance history.

As part of block B64, one or more control actions could be performed by the controller 30 of FIG. 2, or by the external computer device 44 depending on the application. The control actions as contemplated herein pertain to the ultrasonic handpiece 10 and are performed using the recorded capacitance history and other recorded histories from blocks B54-B58. Representative control actions include outputting an SOH indicator indicative of the present health of the ultrasonic handpiece 10. The numeric SOH or an indicator thereof is thus reportable to the user of the ultrasonic handpiece 10 of FIG. 1, or to another interested party such as technical service, possibly via communication with the external computer device 44. The reported numeric SOH could be considered when determining the validity or urgency of certain warranty returns, or the numeric SOH could be used to trigger transmission of an advanced notice or message to the user of the need for imminent or future replacement of the ultrasonic handpiece 10, such as via a text message, email, or letter.

Other control actions contemplated as part of block B64 can result from the real-time characterization of the capacitance values (C₀, C₁) of block B58. For example, the processor 36 could use the capacitance values (C₀, C₁) as feedback for the purpose of adjusting a drive voltage or current of the piezo-crystals 26, and for optimizing the drive frequency (f_{d}). Optimization in this sense would include moving the drive frequency (f_{d}) closer to the calculated resonant frequency (fᵣ), ideally matching the resonant frequency (fᵣ) as closely as possible. Those skilled in the art in view will appreciate that aspects of the foregoing disclosure could be used in other respects beyond reducing warranty costs. For instance, during initial manufacturing, the initial capacitance values that are later updated in real-time according to the method 50 could be used as additional manufacturing test criteria. This would provide an extra level of protection against inadvertent incorporation of faulty piezo-crystals 26 into a given ultrasonic handpiece 10. Build quality would be improved while helping to prevent premature degradation of the ultrasonic handpiece 10 in the field.

Additionally, in markets within which it is common to “flash sanitize” the ultrasonic handpiece 10 in an autoclave, a given ultrasonic handpiece 10 could be reused as much as 30-40 times a day. Such a use case would stand to benefit from the additional insight provided by the present approach into the effects of repeated exposure to steam and heat on the health trajectory of the ultrasonic handpiece 10. These and other possible attendant benefits thus would follow from the disclosed solutions.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

1. A controller for use with an ultrasonic handpiece having a load constructed of at least one piezoelectric drive crystal, comprising:
a processor; and
a computer-readable storage medium (“memory”) on which is recorded an instruction set, wherein execution of the instruction set by the processor causes the processor to:
calculate, during real-time operation of the ultrasonic handpiece, (i) a load capacitance of the load at a calibrated sampling rate, and (ii) a dissipation factor of the load capacitance;
record the load capacitance and the dissipation factor in the memory as a recorded capacitance history;
calculate a numeric state of health (SOH) of the load using the recorded capacitance history; and
execute a control action of the ultrasonic handpiece in response to the numeric SOH.

2. The controller of claim 1, wherein the execution of the instruction set causes the processor to:
selectively adjust a drive frequency of the load as at least part of the control action, such that the drive frequency is moved closer in value to a resonant frequency of the ultrasonic handpiece.

3. The controller of claim 1, wherein the execution of the instruction set by the processor causes the processor to:
output an electronic health signal to an external computer device in response to the numeric SOH, wherein the electronic health signal is representative of a relative health level of the load as indicated by the numeric SOH.

4. The controller of claim 3, wherein the processor is configured to calculate a normalized value of the numeric SOH, and the relative health level is a value on a scale of 0 to 1.

5. The controller of claim 1, wherein the ultrasonic handpiece includes an electrical connector, wherein the processor and the memory are enclosed within the electrical connector.

6. The controller of claim 1, wherein the processor is configured, in response to a request signal from an external computer device, to selectively transmit the recorded capacitance history to the external computer device via a network connection as at least part of the control action.

7. The controller of claim 1, wherein the execution of the instruction set causes the processor to:
periodically determine additional electrical parameters of the ultrasonic handpiece during the real-time operation of the ultrasonic handpiece, the additional electrical parameters including an estimated internal resistance, an estimated internal inductance, and an internal temperature of the ultrasonic handpiece;
record the values of the additional electrical parameters in the memory; and
use the values of the additional electrical parameters in conjunction with the numeric SOH to predict an optimal drive frequency of the ultrasonic handpiece.

8. The controller of claim 1, wherein the execution of the instruction set causes the processor to:
calculate the numeric SOH using a ratio of a baseline dissipation factor to a latest-computed value of the dissipation factor of the load capacitance.

9. A method for evaluating an ultrasonic handpiece having a load constructed of at least one piezoelectric drive crystal, comprising:
calculating, via a processor of a controller, (i) a load capacitance of the load at a calibrated sampling rate, and (ii) a dissipation factor of the load capacitance during real-time operation of the ultrasonic handpiece, wherein the load capacitance and the dissipation factor together form capacitance values;
recording the capacitance values in a computer-readable storage medium (“memory”) of the controller at the calibrated sampling rate as a recorded capacitance history;
calculating a numeric state of health (SOH) of the load via the processor using the recorded capacitance history; and
executing a control action of the ultrasonic handpiece via the processor in response to the numeric SOH, including outputting an electronic health signal to an external computer device in response to the numeric SOH, wherein the electronic health signal is representative of a relative health level of the load as indicated by the numeric SOH.

10. The method of claim 9, further comprising:
recording a first correlation between a temperature increase and the resonant frequency of the ultrasonic handpiece;
recording a second correlation between an aging time and a resonant frequency of the ultrasonic handpiece; and
selectively adjusting a drive frequency of the load from a control console using the first correlation and the second correlation, via the controller, as at least part of the control action, such that the drive frequency is closer in value to a resonant frequency of the ultrasonic handpiece.

11. The method of claim 9, further comprising:
in response to receipt by the controller of a request signal from an external computer device, selectively transmitting the recorded capacitance history to the external device via a network connection.

12. The method of claim 9, further comprising:
periodically calculating a value of at least one additional electrical parameter of the ultrasonic handpiece during the real-time operation of the ultrasonic handpiece;
recording the value of the at least one additional electrical parameter in the memory; and
adjusting the numeric SOH using the value of the at least one additional electrical parameter.

13. The method of claim 12, wherein periodically calculating the value of at least one additional electrical parameter of the ultrasonic handpiece includes periodically estimating an internal resistance and an internal inductance of the ultrasonic handpiece and periodically measuring an internal temperature of the ultrasonic handpiece.

14. An ultrasonic handpiece comprising:
a housing defining a cavity therein;
a working tip connected to the housing;
a load contained within the cavity and constructed of at least one piezoelectric drive crystal, the load being configured to vibrate the working tip at a resonant frequency of the ultrasonic handpiece when the load is activated at a drive frequency; and
a controller connected to the housing and configured to:
calculate a load capacitance of the load and a dissipation factor of the load capacitance, wherein the load capacitance is due to vibration of the ultrasonic handpiece at a calibrated sampling rate during real-time operation of the ultrasonic handpiece, and wherein the load capacitance and the dissipation factor form capacitance values;
record the capacitance values in memory at the calibrated sampling rate as a recorded capacitance history;
calculate a numeric state of health (SOH) of the load using the recorded capacitance history; and
execute a control action of the ultrasonic handpiece in response to the numeric SOH, including selectively outputting an electronic health signal to an external computer device in response to the numeric SOH and selectively adjust the drive frequency such that the drive frequency is closer in value to the resonant frequency of the ultrasonic handpiece, wherein the electronic health signal is representative of a relative health level of the load as indicated by the numeric SOH.

15. The ultrasonic handpiece of claim 14, further comprising:
an electrical connector configured to connect to the ultrasonic handpiece, wherein the processor and the memory are enclosed within the electrical connector.
